(19) United States
(12) Patent Application Publication
(10) Pub. No.: US 2026/0290552 A1
(43) Pub. Date: Sep. 24, 2026

(54) SYSTEMS, DEVICES AND METHODS FOR IMPROVED MEAL AND THERAPY INTERFACES IN ANALYTE MONITORING SYSTEMS

(71) Applicant: ABBOTT DIABETES CARE INC., Alameda, CA (US)

(72) Inventor: Gary A. Hayter, Oakland, CA (US)

(21) Appl. No.: 19/689,695

(22) Filed: May 27, 2026

Related U.S. Application Data
(63) Continuation of application No. 17/363,935, filed on Jun. 30, 2021.
Application No. 17/363,935 is a continuation of application No. PCT/US20/12134, filed on Jan. 3, 2020.
(60) Provisional application No. 62/788,310, filed on Jan. 4, 2019.

(51) Int. Cl. G16H 20/60; A61B 5/145; G16H 20/10

(52) U.S. Cl. CPC G16H 20/60; A61B 5/14532; G16H 20/10

(57) ABSTRACT

Various embodiments of systems, devices and methods for improved meal and therapy interfaces in analyte monitoring systems are disclosed. These embodiments can determine a medication dosage to be administered with the consumption of a meal, identify meal start and meal peak response candidates, and recommend user-initiated analyte checks.

### CROSS-REFERENCE TO RELATED APPLICATIONS

This application is a continuation of U.S. patent application Ser. No. 17/363,935, filed Jun. 30, 2021, which is a continuation of International Patent Application No. PCT/US20/12134, filed Jan. 3, 2020, which claims priority to U.S. Provisional Application No. 62/788,310, filed Jan. 4, 2019, all of which are herein expressly incorporated by reference in their entireties for all purposes.

### FIELD

The subject matter described herein relates generally to systems, devices, and methods for improved meal and therapy interfaces in analyte monitoring systems. In particular, embodiments are provided for determining a medication dosage to be administered with the consumption of a meal, identifying meal start and meal peak response candidates, and recommending user-initiated analyte checks.

### BACKGROUND

The increased prevalence of Type 2 diabetes and Metabolic Syndrome over the past few decades can be been attributed to changing diet and activity levels. For example, consumption of more readily available high glycemic index foods can cause rapid post-prandial increase of blood glucose and insulin levels, which has a positive association with weight gain and obesity. These conditions can be further traced to an increased risk of developing these and other diseases.

Most people generally understand the importance of their diet. However, in practice, many individuals struggle with translating this general awareness to their specific food choices. These problems exist primarily because people cannot directly see the impact of their choices. This can lead to misconceptions about portion size, misunderstandings about which foods are relatively healthy, and a general lack of awareness regarding the necessary duration and intensity of activity for maintaining good health. These problems are further exacerbated by advertisements, habits, peer pressure, food preferences, and recommendations based on overgeneralizations.

To address these issues, an individual's physiological responses can be tracked and better understood by analyte monitoring systems. Because high glucose levels are primarily driven by the consumption of food, the level of post-prandial glucose can relate to the amount of carbohydrates and other meal components consumed by the individual, as well as to the individual's physiological response to meals. However, a challenge for the analysis of this influx of data is to represent the data in a meaningful manner that enables efficient action. Data relating to meal selection, and the subsequent impact, should be understood on a clinical basis, as well as a personal basis for the individual, the meal administrator, and/or the medical professional to understand and moderate glucose excursions, such as episodes of hyperglycemia.

Prior solutions for correlating an individual's analyte data with meal consumption, as well as pre-prandial and post-prandial responses, suffer from numerous deficiencies. For example, some systems require that the individual perform numerous inconvenient and uncomfortable discrete blood glucose measurements (e.g., finger stick blood glucose tests). These solutions can also suffer from an insufficient number of data points to adequately determine a glycemic response to a meal. For example, an individual may perform a discrete blood glucose measurement at a time before or after the individual's glycemic response peaks, making it difficult to accurately ascertain the glycemic response, and to meaningfully compare meals based on the glycemic response. A deficiency in data points can also make it difficult to automatically detect the start of a meal event in the individual's analyte data.

Moreover, some prior and existing systems place significant reliance upon manual logging of meals by the individual, which can be unreliable. Another approach to determining pre-prandial and post-prandial meal responses involves collecting dense glucose measurements within a pre-determined time of day window, where glucose values within the window are assumed to represent pre-breakfast and/or post-breakfast times, for example. However, with respect to this approach, the reliability of the estimates will largely depend upon the consistency in the patient's meal timing routine, which can also be unreliable.

Other prior systems seek to detect meal events based simply on the existence of a rise in glucose levels, such as those described in U.S. Publication No. 2003/0208113. These systems, however, can be inadequate because they fail to take into account the individual's prior meal history, and can overestimate the number of meals that an individual has consumed.

A related challenge concerns the determination of a medication dosage (e.g., an insulin dosage) for diabetic individuals to compensate for an anticipated glycemic rise that occurs after consumption of a meal. This dose is often referred to as a meal bolus. Determining the appropriate amount of insulin to be administered can be difficult, and typically entails using a prior art bolus calculator that relies on parameters such as an individual's insulin sensitivity, the individual's insulin on-board, and the amount of carbohydrates in the meal. The carbohydrate content for home-cooked meals, for example, can be difficult to determine as it is often based on the amount of each individual ingredient in the recipe and may require the user to make estimates based on the weight of various portions of the meals. It also requires a carbohydrate determination to be made for each part of the meal. For example, in the case of a dinner including meat, casserole, and a vegetable, carbohydrate content must be determined for each component separately and then summed together for entry into the bolus calculator. The time and effort required in making such calculations can be particularly burdensome to diabetics and often result in the diabetic guessing as to the carbohydrate content.

For these and other reasons, needs exist for improved meal and therapy interfaces for analyte monitoring systems.

### SUMMARY

Example embodiments of systems, devices, and methods are described herein for improved meal and therapy interfaces for use in vivo analyte monitoring systems. These embodiments can provide for systems, devices, and methods for determining a medication dosage to be administered with consumption of a meal, identifying meal start and meal peak response candidates, and recommending user-initiated analyte checks.

According to one embodiment, for example, a computer-implemented method for determining a medication dosage for administration with the consumption of a meal includes the steps of receiving a user-inputted entry associated with a meal, referencing a first database to determine one or more nutrient parameters associated with the meal, identifying a closest-matched meal in a second database based on the nutrient parameters, and determining a medication dosage associated with the closest-matched meal.

According to another embodiment, a computer-implemented method for identifying a set of meal start candidates and meal peak response candidates includes the steps of determining time derivatives for data points corresponding to a monitored analyte level, creating a set of meal start candidates and meal peak response candidates by determining an optima of acceleration based on the time derivatives, retrieving multiple user-initiated checks and grouping the checks into time clusters, determining a time cluster start point, a time cluster end point, and a time cluster central tendency point for each time cluster, and removing a subset of meal start candidates from the set, wherein the subset includes one or more meal start candidates that are not within a predetermined temporal range of either a time cluster start point or a time cluster end point.

According to yet another embodiment, a computer-implemented method for recommending a user-initiated analyte check includes the steps of receiving a recorded action by a user, evaluating a historical log to determine if the recorded action corresponds to a historical user action associated with a glycemic risk, in response to determining that the recorded action corresponds to the historical user action associated with the glycemic risk, calculating an elapsed time until reaching an actionable time period associated with the glycemic risk, and outputting a notification to the user to perform a user-initiated analyte check after the elapsed time.

Numerous examples of algorithms and methods for performing combinations and/or variations of one or both of these detection mechanisms are provided, as well as example embodiments of systems and devices for performing the same.

Other systems, devices, methods, features and advantages of the subject matter described herein will be or will become apparent to one with skill in the art upon examination of the following figures and detailed description. It is intended that all such additional systems, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is an illustrative view depicting an example embodiment of an in vivo analyte monitoring system.

FIG. 2 is a block diagram of an example embodiment of a reader device.

FIG. 3 is a block diagram of an example embodiment of a sensor control device.

FIG. 4 is a block diagram of an example embodiment of a system architecture configured to determine a medication dosage to be administered with the consumption of a meal.

FIG. 5 is a flow diagram depicting an example embodiment of a method for determining a medication dosage to be administered with the consumption of a meal.

FIGS. 6A to 6C are graphs depicting distributions of user-initiated analyte checks.

FIGS. 7A and 7B are graphs depicting various analyte measurements and characteristics thereof.

FIG. 8 is a flow diagram depicting an example embodiment of a method for determining a set of meal start and meal peak response candidates.

FIGS. 9A to 9C are flow diagrams depicting another example embodiment of a method for determining a set of meal start and meal peak response candidates.

FIG. 10 is a flow diagram depicting an example embodiment of a method for recommending a user-initiated analyte check.

FIG. 11 is a flow diagram depicting another example embodiment of a method for recommending a user-initiated analyte check.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

The publications discussed herein are provided solely for their disclosure prior to the filing date of the present application. Nothing herein is to be construed as an admission that the present disclosure is not entitled to antedate such publications by virtue of prior disclosure. Furthermore, the dates of publication provided may be different from the actual publication dates which may need to be independently confirmed.

Generally, embodiments of the present disclosure are used with systems, devices, and methods for detecting at least one analyte, such as glucose, in a bodily fluid (e.g., subcutaneously within the interstitial fluid (“ISF”) or blood, within the dermal fluid of the dermal layer, or otherwise). Accordingly, many embodiments include in vivo analyte sensors structurally configured so that at least a portion of the sensor is, or can be, positioned in the body of a user to obtain information about at least one analyte of the body. However, the embodiments disclosed herein can be used with in vivo analyte monitoring systems that incorporate in vitro capability, as well as purely in vitro or ex vivo analyte monitoring systems, including those systems that are entirely non-invasive.

Furthermore, for each and every embodiment of a method disclosed herein, systems and devices capable of performing each of those embodiments are covered within the scope of the present disclosure. For example, embodiments of electronic systems are disclosed, and these electronic systems can include non-transitory memory (e.g., for storing instructions), processing circuitry (e.g., for executing instructions), power sources, communication circuitry, transmitters, receivers, and/or controllers that can perform any and all method steps or facilitate the execution of any and all method steps.

A number of embodiments of the present disclosure are designed to improve upon the computer-implemented capabilities of analyte monitoring systems with respect to meal and therapy interfaces. In some embodiments, for example, a medication dosage for administration with the consumption of a meal can be determined by identifying a closest-matched meal in a database based on certain nutrient parameters. These embodiments can improve the accuracy of dosage determination software, for example, by referencing an individual's own historical glycemic responses and medication dosages, instead of relying upon an individual's guesswork as to the nutritional content of a meal.

According to other embodiments, data indicative of a monitored analyte level analyte is received from an analyte sensor and can be used by processing circuitry to identify a set of meal start and meal peak response candidates. These embodiments can improve upon the accuracy of software for determining meal start times and meal peak response times, without having to rely upon user estimation or strict adherence to daily meal routines. Further, these embodiments can present a limited and more accurate set of meal start and meal peak response candidates via a graphical interface, which allows the user to more efficiently navigate analyte data collected by an analyte monitoring system.

According to still other embodiments, if a current recorded action by a user is determined to have an associated glycemic risk, a recommendation to perform a user-initiated analyte check (e.g., a sensor scan) can be outputted to the user after an elapsed time. These embodiments evaluate a historical log of the user's past actions and associated glycemic risk to determine whether a future user-initiated analyte check is warranted. In this regard, these embodiments improve upon analyte monitoring systems by increasing and/or maintaining user engagement of the system through interactive user interfaces, as compared to known systems with passive interfaces.

Accordingly, the embodiments described herein reflect various computer-implemented improvements over prior analyte monitoring systems, and their respective user interfaces, in many respects. In particular, these embodiments improve upon the accuracy of analyte monitoring systems with respect to medication dosage determination, meal start and meal peak response detection, and glycemic risk determinations. Further, the embodiments described herein utilize specific types of data (e.g., user-initiated analyte check information) in a non-conventional way. Other features and advantages of the disclosed embodiments are further discussed below.

Before describing the embodiments in detail, however, it is first desirable to describe examples of devices that can be present within, for example, an in vivo analyte monitoring system, as well as examples of their operation, all of which can be used with the embodiments described herein.

### Example Embodiments of Analyte Monitoring Systems

There are various types of analyte monitoring systems. “Continuous Analyte Monitoring” systems (or “Continuous Glucose Monitoring” systems), for example, are in vivo systems that can transmit data from a sensor control device to a reader device repeatedly or continuously without prompting, e.g., automatically according to a schedule. “Flash Analyte Monitoring” systems (or “Flash Glucose Monitoring” systems or simply “Flash” systems), as another example, are in vivo systems that can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo monitoring systems can include a sensor that, while positioned in vivo, makes contact with the bodily fluid of the user and senses one or more analyte levels contained therein. The sensor can be part of a sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few. As used herein, these terms are not limited to devices with analyte sensors, and encompass devices that have sensors of other types, whether biometric or non-biometric. The term “on body” refers to any device that resides directly on the body or in close proximity to the body, such as a wearable device (e.g., glasses, watch, wristband or bracelet, neckband or necklace, etc.).

In vivo monitoring systems can also include one or more reader devices that receive sensed analyte data from the sensor control device. These reader devices can process and/or display the sensed analyte data, or sensor data, in any number of forms, to the user. These devices, and variations thereof, can be referred to as “handheld reader devices,” “reader devices” (or simply, “readers”), “handheld electronics” (or handhelds), “portable data processing” devices or units, “data receivers,” “receiver” devices or units (or simply receivers), “relay” devices or units, or “remote” devices or units, to name a few. Other devices such as personal computers have also been utilized with or incorporated into in vivo and in vitro monitoring systems.

In vivo analyte monitoring systems can be differentiated from “in vitro” systems that contact a biological sample outside of the body (or rather “ex vivo”) and that typically include a meter device that has a port for receiving an analyte test strip carrying a bodily fluid of the user, which can be analyzed to determine the user's analyte level. As mentioned, the embodiments described herein can be used with in vivo systems, in vitro systems, and combinations thereof.

The embodiments described herein can be used to monitor and/or process information regarding any number of one or more different analytes. Analytes that may be monitored include, but are not limited to, acetyl choline, amylase, bilirubin, cholesterol, chorionic gonadotropin, glycosylated hemoglobin (HbAlc), creatine kinase (e.g., CK-MB), creatine, creatinine, DNA, fructosamine, glucose, glucose derivatives, glutamine, growth hormones, hormones, ketones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In embodiments that monitor more than one analyte, the analytes may be monitored at the same or different times.

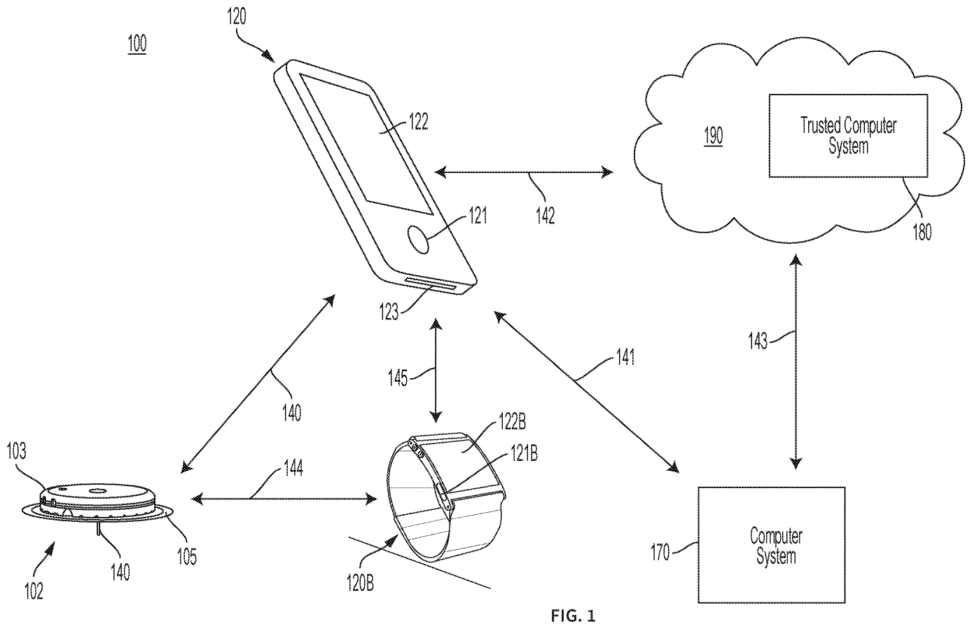

FIG. 1 is an illustrative view depicting an example embodiment of an in vivo analyte monitoring system 100 having a sensor control device 102 and a reader device 120 that communicate with each other over a local communication path (or link) 140, which can be wired or wireless, and uni-directional or bi-directional. According to some embodiments, in vivo monitoring system 100 can also include wearable electronics 120B, such as a smart watch, that can communicate with sensor control device 102 over communication path (or link) 144 and/or reader device 120 over communication path (or link) 145. Communication paths 144 and 145 can be wired or wireless, and uni-directional or bi-directional. In embodiments where paths 140, 144, and 145 are wireless, a near field communication (NFC) protocol, RFID protocol, Bluetooth or Bluetooth Low Energy protocol, Wi-Fi protocol, proprietary protocol, or the like can be used, including those communication protocols in existence as of the date of this filing or their later developed variants.

Reader device 120 is also capable of wired, wireless, or combined communication with a computer system 170 (e.g., a local or remote computer system) over communication path (or link) 141 and with a network 190, such as the internet or the cloud, over communication path (or link) 142. Communication with network 190 can involve communication with trusted computer system 180 within network 190, or though network 190 to computer system 170 via communication link (or path) 143. Communication paths 141, 142, and 143 can be wireless, wired, or both, can be uni-directional or bi-directional, and can be part of a telecommunications network, such as a Wi-Fi network, a local area network (LAN), a wide area network (WAN), the internet, or other data network. In some cases, communication paths 141 and 142 can be the same path. All communications over paths 140, 141, and 142 can be encrypted and sensor control device 102, reader device 120, computer system 170, and trusted computer system 180 can each be configured to encrypt and decrypt those communications sent and received.

Variants of devices 102 and 120, as well as other components of an in vivo-based analyte monitoring system that are suitable for use with the system, device, and method embodiments set forth herein, are described in U.S. Publication No. 2011/0213225 (the '225 Publication), which is incorporated by reference herein in its entirety for all purposes.

Sensor control device 102 can include a housing 103 containing in vivo analyte monitoring circuitry and a power source. In this embodiment, the in vivo analyte monitoring circuitry is electrically coupled with an analyte sensor 104 that extends through an adhesive patch 105 and projects away from housing 103. Adhesive patch 105 contains an adhesive layer (not shown) for attachment to a skin surface of the body of the user. Other forms of body attachment to the body may be used, in addition to or instead of adhesive.

Sensor 104 is adapted to be at least partially inserted into the body of the user, where it can make fluid contact with that user's bodily fluid (e.g., subcutaneous (subdermal) fluid, dermal fluid, or blood) and be used, along with the in vivo analyte monitoring circuitry, to measure analyte-related data of the user. Sensor 104 and any accompanying sensor control electronics can be applied to the body in any desired manner. For example, an insertion device (not shown) can be used to position all or a portion of analyte sensor 104 through an external surface of the user's skin and into contact with the user's bodily fluid. In doing so, the insertion device can also position sensor control device 102 with adhesive patch 105 onto the skin. In other embodiments, insertion device can position sensor 104 first, and then accompanying sensor control electronics can be coupled with sensor 104 afterwards, either manually or with the aid of a mechanical device. Examples of insertion devices are described in U.S. Publication Nos. 2008/0009692, 2011/0319729, 2015/0018639, 2015/0025345, and 2015/0173661, all which are incorporated by reference herein in their entireties and for all purposes.

After collecting raw data from the user's body, sensor control device 102 can apply analog signal conditioning to the data and convert the data into a digital form of the conditioned raw data. In some embodiments, sensor control device 102 can then algorithmically process the digital raw data into a form that is representative of the user's measured biometric (e.g., analyte level) and/or one or more analyte metrics based thereupon. For example, sensor control device 102 can include processing circuitry to calculate analyte metrics and algorithmically perform any of the method steps described herein. Sensor control device 102 can then encode and wirelessly communicate the calculated analyte metrics, processed sensor data, notifications, or any other data to reader device 120 and/or wearable electronics 120B, which in turn can format or graphically process the received data for digital display to the user. In other embodiments, in addition to, or in lieu of, wirelessly communicating sensor data to another device (e.g., reader device 120 and/or wearable electronics 120B), sensor control device 102 can graphically process the final form of the data such that it is ready for display, and display that data on a display of sensor control device 102. In some embodiments, the final form of the biometric data (prior to graphic processing) is used by the system (e.g., incorporated into a diabetes monitoring regime) without processing for display to the user.

In still other embodiments, the conditioned raw digital data can be encoded for transmission to another device, e.g., reader device 120 and/or wearable electronics 120B, which then algorithmically processes that digital raw data into a form representative of the user's measured biometric (e.g., a form readily made suitable for display to the user) and/or one or more analyte metrics based thereupon. Reader device 120 and/or wearable electronics 120B can include processing circuitry to calculate analyte metrics and algorithmically perform any of the method steps described herein. This algorithmically processed data can then be formatted or graphically processed for digital display to the user.

In other embodiments, sensor control device 102 and reader device 120 transmit the digital raw data to another computer system for algorithmic processing and display.

Reader device 120 can include a display 122 to output information to the user and/or to accept an input from the user, and an optional input component 121 (or more), such as a button, actuator, touch sensitive switch, capacitive switch, pressure sensitive switch, jog wheel or the like, to input data, commands, or otherwise control the operation of reader device 120. In certain embodiments, display 122 and input component 121 may be integrated into a single component, for example, where the display can detect the presence and location of a physical contact touch upon the display, such as a touch screen user interface. In certain embodiments, input component 121 of reader device 120 may include a microphone and reader device 120 may include software configured to analyze audio input received from the microphone, such that functions and operation of the reader device 120 may be controlled by voice commands. In certain embodiments, an output component of reader device 120 includes a speaker (not shown) for outputting information as audible signals. Similar voice responsive components such as a speaker, microphone and software routines to generate, process and store voice driven signals may be included in sensor control device 102. According to some embodiments, wearable electronics 120B can include components, including a display 122B (that can have a touch screen user interface), and an optional input component 121B, that function in a manner similar to like components of reader device 120.

Reader device 120 can also include one or more data communication ports 123 for wired data communication with external devices such as computer system 170 or sensor control device 102. Example data communication ports include USB ports, mini USB ports, USB Type-C ports, USB micro-A and/or micro-B ports, RS-232 ports, Ethernet ports, Firewire ports, or other similar data communication ports configured to connect to the compatible data cables. Reader device 120 may also include an integrated or attachable in vitro glucose meter, including an in vitro test strip port (not shown) to receive an in vitro glucose test strip for performing in vitro blood glucose measurements.

Reader device 120 and/or wearable electronics 120B can display the measured biometric data wirelessly received from sensor control device 102 and can also be configured to output alarms, alert notifications, glucose values, etc., which may be visual, audible, tactile, or any combination thereof. Further details and other display embodiments can be found in, e.g., U.S. Publication No. 2011/0193704, which is incorporated herein by reference in its entirety for all purposes.

Reader device 120 can function as a data conduit to transfer the measured data and/or analyte metrics from sensor control device 102 to computer system 170 or trusted computer system 180. In certain embodiments, the data received from sensor control device 102 may be stored (permanently or temporarily) in one or more memories of reader device 120 prior to uploading to system 170, 180 or network 190.

Computer system 170 may be a personal computer, a server terminal, a laptop computer, a tablet, or other suitable data processing device. Computer system 170 can be (or include) software for data management and analysis and communication with the components in analyte monitoring system 100. Computer system 170 can be used by the user or a medical professional to display and/or analyze the biometric data measured by sensor control device 102. In some embodiments, sensor control device 102 can communicate the biometric data directly to computer system 170 without an intermediary such as reader device 120, or indirectly using an internet connection (also optionally without first sending to reader device 120). Operation and use of computer system 170 is further described in the '225 Publication incorporated herein. Analyte monitoring system 100 can also be configured to operate with a data processing module (not shown), also as described in the incorporated '225 Publication.

Trusted computer system 180 can be within the possession of the manufacturer or distributor of sensor control device 102, either physically or virtually through a secured connection, and can be used to perform authentication of sensor control device 102, for secure storage of the user's biometric data, and/or as a server that serves a data analytics program (e.g., accessible via a web browser) for performing analysis on the user's measured data.

### Example Embodiments of Reader Devices

Reader device 120 can be a mobile communication device such as a dedicated reader device (configured for communication with a sensor control device 102, and optionally a computer system 170, but without mobile telephony communication capability) or a mobile telephone including, but not limited to, a Wi-Fi or internet enabled smart phone, tablet, or personal digital assistant (PDA). Examples of smart phones can include those mobile phones based on a Windows® operating system, Android™ operating system, iPhone® operating system, Palm® WebOS™, Blackberry® operating system, or Symbian® operating system, with data network connectivity functionality for data communication over an internet connection and/or a local area network (LAN).

Reader device 120 can also be configured as a mobile smart wearable electronics assembly, such as an optical assembly that is worn over or adjacent to the user's eye (e.g., a smart glass or smart glasses, such as Google glasses, which is a mobile communication device). This optical assembly can have a transparent display that displays information about the user's analyte level (as described herein) to the user while at the same time allowing the user to see through the display such that the user's overall vision is minimally obstructed. The optical assembly may be capable of wireless communications similar to a smart phone. Other examples of wearable electronics include devices that are worn around or in the proximity of the user's wrist (e.g., a watch, etc.), neck (e.g., a necklace, etc.), head (e.g., a headband, hat, etc.), chest, or the like. According to some embodiments, for example, wearable electronics can include a smart watch 120B, as shown in FIG. 1, that is capable of transmitting and receiving data directly from sensor control device 102 over communication path 144 and/or reader device 120 over communication path 145. Additionally, in some embodiments, wearable electronics 120B can include processing circuitry coupled to memory for storing instructions that, when executed by the processing circuitry of wearable electronics 120B, causes the processing circuitry to execute a program for generating an output, such as displaying data indicative of a sensed analyte level on a user interface on the display 122B of wearable electronics, or outputting an auditory or vibratory alert. In some embodiments, data indicative of a sensed analyte level can be received by wearable electronics 120 from either or both of the sensor control device 102 or reader device 120.

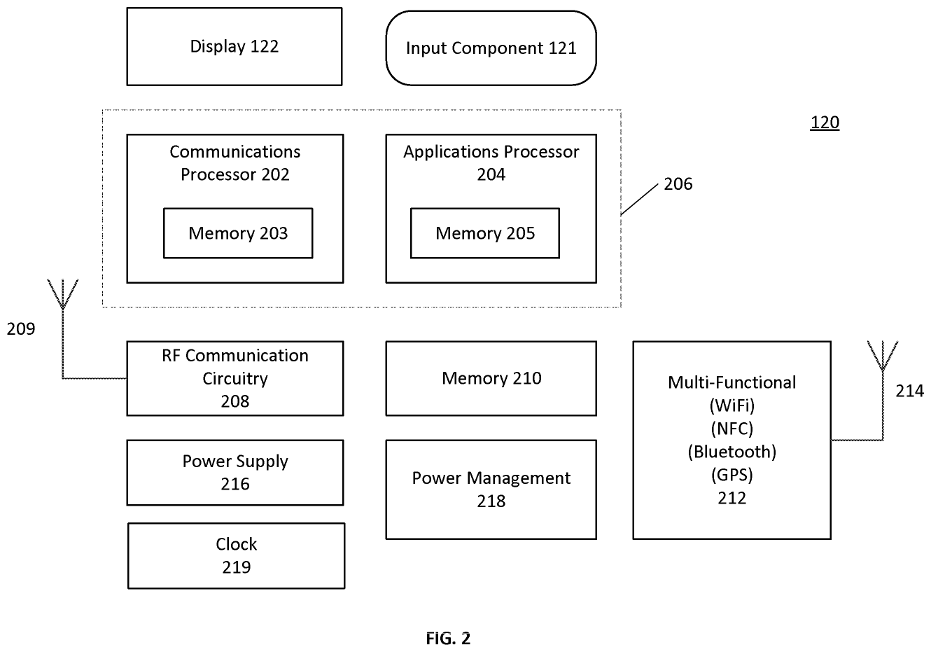

FIG. 2 is a block diagram of an example embodiment of a reader device 120 configured as a smart phone. Here, reader device 120 includes an input component 121, display 122, and processing circuitry 206, which can include one or more processors, microprocessors, controllers, and/or microcontrollers, each of which can be a discrete chip or distributed amongst (and a portion of) a number of different chips. Here, processing circuitry 206 includes a communications processor 202 having on-board memory 203 and an applications processor 204 having on-board memory 205. Reader device 120 further includes RF communication circuitry 208 coupled with an RF antenna 209, a memory 210, multi-functional circuitry 212 with one or more associated antennas 214, a power supply 216, power management circuitry 218, and a clock 219. FIG. 2 is an abbreviated representation of the typical hardware and functionality that resides within a smart phone and those of ordinary skill in the art will readily recognize that other hardware and functionality (e.g., codecs, drivers, glue logic) can also be included.

Communications processor 202 can interface with RF communication circuitry 208 and perform analog-to-digital conversions, encoding and decoding, digital signal processing and other functions that facilitate the conversion of voice, video, and data signals into a format (e.g., in-phase and quadrature) suitable for provision to RF communication circuitry 208, which can then transmit the signals wirelessly. Communications processor 202 can also interface with RF communication circuitry 208 to perform the reverse functions necessary to receive a wireless transmission and convert it into digital data, voice, and video. RF communication circuitry 208 can include a transmitter and a receiver (e.g., integrated as a transceiver) and associated encoder logic.

Applications processor 204 can be adapted to execute the operating system and any software applications that reside on reader device 120, process video and graphics, and perform those other functions not related to the processing of communications transmitted and received over RF antenna 209. The smart phone operating system will operate in conjunction with a number of applications on reader device 120. Any number of applications (also known as “user interface applications”) can be running on reader device 120 at any one time, and may include one or more applications that are related to a diabetes monitoring regime, in addition to the other commonly used applications that are unrelated to such a regime, e.g., email, calendar, weather, sports, games, etc. For example, the data indicative of a sensed analyte level and in vitro blood analyte measurements received by the reader device can be securely communicated to user interface applications residing in memory 210 of reader device 120. Such communications can be securely performed, for example, through the use of mobile application containerization or wrapping technologies. In addition, according to some embodiments, reader device 120 can also include an application for communicating data indicative of a sensed analyte level with wearable electronics 120B.

Memory 210 can be shared by one or more of the various functional units present within reader device 120, or can be distributed amongst two or more of them (e.g., as separate memories present within different chips). Memory 210 can also be a separate chip of its own. Memories 203, 205, and 210 are non-transitory, and can be volatile (e.g., RAM, etc.) and/or non-volatile memory (e.g., ROM, flash memory, F-RAM, etc.).

Multi-functional circuitry 212 can be implemented as one or more chips and/or components (e.g., transmitter, receiver, transceiver, and/or other communication circuitry) that perform other functions such as local wireless communications, e.g., with sensor control device 102 under the appropriate protocol (e.g., Wi-Fi, Bluetooth, Bluetooth Low Energy, Near Field Communication (NFC), Radio Frequency Identification (RFID), proprietary protocols, and others) and determining the geographic position of reader device 120 (e.g., global positioning system (GPS) hardware). One or more other antennas 214 are associated with the functional circuitry 212 as needed to operate with the various protocols and circuits.

Power supply 216 can include one or more batteries, which can be rechargeable or single-use disposable batteries. Power management circuitry 218 can regulate battery charging and power supply monitoring, boost power, perform DC conversions, and the like.

Reader device 120 can also include or be integrated with a drug (e.g., insulin, etc.) delivery device such that they, e.g., share a common housing. Examples of such drug delivery devices can include medication pumps having a cannula that remains in the body to allow infusion over a multi-hour or multi-day period (e.g., wearable pumps for the delivery of basal and bolus insulin). Reader device 120, when combined with a medication pump, can include a reservoir to store the drug, a pump connectable to transfer tubing, and an infusion cannula. The pump can force the drug from the reservoir, through the tubing and into the diabetic's body by way of the cannula inserted therein. Other examples of drug delivery devices that can be included with (or integrated with) reader device 120 include portable injection devices that pierce the skin only for each delivery and are subsequently removed (e.g., insulin pens). A reader device 120, when combined with a portable injection device, can include an injection needle, a cartridge for carrying the drug, an interface for controlling the amount of drug to be delivered, and an actuator to cause injection to occur. The device can be used repeatedly until the drug is exhausted, at which point the combined device can be discarded, or the cartridge can be replaced with a new one, at which point the combined device can be reused repeatedly. The needle can be replaced after each injection.

The combined device can function as part of a closed-loop system (e.g., an artificial pancreas system requiring no user intervention to operate) or semi-closed loop system (e.g., an insulin loop system requiring seldom user intervention to operate, such as to confirm changes in dose). For example, the diabetic's analyte level can be monitored in a repeated automatic fashion by sensor control device 102, which can then communicate that monitored analyte level to reader device 120, and the appropriate drug dosage to control the diabetic's analyte level can be automatically determined and subsequently delivered to the diabetic's body. Software instructions for controlling the pump and the amount of insulin delivered can be stored in the memory of reader device 120 and executed by the reader device's processing circuitry. These instructions can also cause calculation of drug delivery amounts and durations (e.g., a bolus infusion and/or a basal infusion profile) based on the analyte level measurements obtained directly or indirectly from sensor control device 102. In some embodiments sensor control device 102 can determine the drug dosage and communicate that to reader device 120.

Example Embodiments of Sensor Control Devices

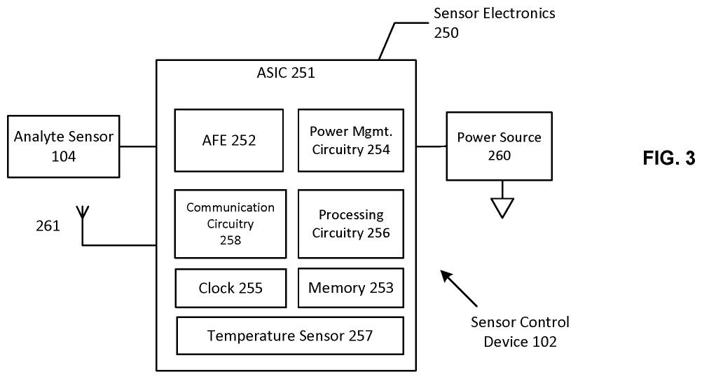

FIG. 3 is a block diagram depicting an example embodiment of sensor control device 102 having analyte sensor 104 and sensor electronics 250 (including analyte monitoring circuitry) that can have the majority of the processing capability for rendering end-result data suitable for display to the user. In FIG. 3, a single semiconductor chip 251 is depicted that can be a custom application specific integrated circuit (ASIC). Shown within ASIC 251 are certain high-level functional units, including an analog front end (AFE) 252, power management (or control) circuitry 254, processing circuitry 256, and communication circuitry 258 (which can be implemented as a transmitter, receiver, transceiver, passive circuit, or otherwise according to the communication protocol). In this embodiment, both AFE 252 and processing circuitry 256 are used as analyte monitoring circuitry, but in other embodiments either circuit can perform the analyte monitoring function. Processing circuitry 256 can include one or more processors, microprocessors, controllers, and/or microcontrollers, each of which can be a discrete chip or distributed amongst (and a portion of) a number of different chips.

A memory 253 is also included within ASIC 251 and can be shared by the various functional units present within ASIC 251, or can be distributed amongst two or more of them. Memory 253 can also be a separate chip. Memory 253 is non-transitory and can be volatile and/or non-volatile memory. In this embodiment, ASIC 251 is coupled with power source 260, which can be a coin cell battery, or the like. AFE 252 interfaces with in vivo analyte sensor 104 and receives measurement data therefrom and outputs the data to processing circuitry 256 in digital form, which in turn can, in some embodiments, process in any of the manners described elsewhere herein. This data can then be provided to communication circuitry 258 for sending, by way of antenna 261, to reader device 120 (not shown), for example, where minimal further processing is needed by the resident software application to display the data. Antenna 261 can be configured according to the needs of the application and communication protocol. Antenna 261 can be, for example, a printed circuit board (PCB) trace antenna, a ceramic antenna, or a discrete metallic antenna. Antenna 261 can be configured as a monopole antenna, a dipole antenna, an F-type antenna, a loop antenna, and others.

Information may be communicated from sensor control device 102 to a second device (e.g., reader device 120) at the initiative of sensor control device 102 or reader device 120. For example, information can be communicated automatically and/or repeatedly (e.g., continuously) by sensor control device 102 when the analyte information is available, or according to a schedule (e.g., about every 1 minute, about every 5 minutes, about every 10 minutes, or the like), in which case the information can be stored or logged in a memory of sensor control device 102 for later communication. The information can be transmitted from sensor control device 102 in response to receipt of a request by the second device. This request can be an automated request, e.g., a request transmitted by the second device according to a schedule, or can be a request generated at the initiative of a user (e.g., an ad hoc or manual request, or a “user-initiated analyte check”). In some embodiments, a manual request for data is referred to as a “scan” of sensor control device 102 or an “on-demand” data transfer from device 102. In some embodiments, the second device can transmit a polling signal or data packet to sensor control device 102, and device 102 can treat each poll (or polls occurring at certain time intervals) as a request for data and, if data is available, then can transmit such data to the second device. In many embodiments, the communication between sensor control device 102 and the second device are secure (e.g., encrypted and/or between authenticated devices), but in some embodiments the data can be transmitted from sensor control device 102 in an unsecured manner, e.g., as a broadcast to all listening devices in range.

Different types and/or forms and/or amounts of information may be sent as part of each communication including, but not limited to, one or more of current sensor measurements (e.g., the most recently obtained analyte level information temporally corresponding to the time the reading is initiated), rate of change of the measured metric over a predetermined time period, rate of the rate of change of the metric (acceleration in the rate of change), or historical metric information corresponding to metric information obtained prior to a given reading and stored in a memory of sensor control device 102.

Some or all of real time, historical, rate of change, rate of rate of change (such as acceleration or deceleration) information may be sent to reader device 120 in a given communication or transmission. In certain embodiments, the type and/or form and/or amount of information sent to reader device 120 may be preprogrammed and/or unchangeable (e.g., preset at manufacturing), or may not be preprogrammed and/or unchangeable so that it may be selectable and/or changeable in the field one or more times (e.g., by activating a switch of the system, etc.). Accordingly, in certain embodiments reader device 120 can output a current (real time) sensor-derived analyte value (e.g., in numerical format), a current rate of analyte change (e.g., in the form of an analyte rate indicator such as an arrow pointing in a direction to indicate the current rate), and analyte trend history data based on sensor readings acquired by and stored in memory of sensor control device 102 (e.g., in the form of a graphical trace). Additionally, an on-skin or sensor temperature reading or measurement may be collected by an optional temperature sensor 257. Those readings or measurements can be communicated (either individually or as an aggregated measurement over time) from sensor control device 102 to another device (e.g., reader 120). The temperature reading or measurement, however, may be used in conjunction with a software routine executed by reader device 120 to correct or compensate the analyte measurement output to the user, instead of or in addition to actually displaying the temperature measurement to the user.

### Example Embodiments for Determining a Medication Dosage to Be Administered With a Meal

Example embodiments of systems, devices, and methods for determining a medication dosage to be administered with the consumption of a meal will now be described. As described earlier, certain individuals, such as those with diabetes, need to compensate for an anticipated glycemic rise occurring after the consumption of a meal by administering medication, such as insulin. The medication dosage is often referred to as a meal bolus because it is an infusion of medication for the purpose of compensating for a meal.

Some prior systems and methods for determining a medication dosage to be administered with the consumption of a meal require an individual to manually count or estimate carbohydrates. These systems can lead to inaccurate and inconsistent medication dosages, as it can be difficult for individuals to accurately estimate the amount of carbohydrates and other nutritional components in their food. In addition, glycemic responses to nutrients can vary from individual to individual, as it is unlikely that different individuals all respond to the same nutrients the same way.

Other systems and methods have attempted to address this challenge by recording repeated instances of meal consumption in a database, along with descriptions of the meals and associated medication dosages. Corresponding analyte data (e.g., post-prandial glucose data) from an analyte monitoring system, such as an in vivo analyte monitoring system, can be also associated with records in the database based on a time period corresponding to the consumption of the meal. Association of the meal with analyte data from prior instances where medication dosages were administered can enable the individual or a health care provider (HCP) to readily identify beneficial medication titrations to improve future glycemic responses. These systems and methods are further described in U.S. patent application Ser. No. 15/863,279, now U.S. Publ. No. 2018/0197628, which is incorporated by reference herein in its entirety and for all purposes.

The embodiments described herein reflect improvements to the aforementioned systems and methods. For example, the embodiments described herein can determine a medication dosage to be administered with the consumption of a meal that an individual has not consumed before. At a general level, the example embodiments allow the individual to input meal information into an interface and, based on various nutritional parameters associated with the meal, a proper bolus amount for the meal is determined. More specifically, the example embodiment methods described herein include the steps of receiving a user-inputted entry associated with a new meal, referencing a first database to determine the nutritional content of the new meal, matching the new meal to a closest-matched meal in a second database based on the nutritional content, and determining a medication dosage associated with the closest-matched meal.

Because the embodiments are based in part on an individual's typical experience, they can be referred to herein as “experiential” tools. For ease of discussion, the example embodiments will be described in the context of insulin bolus dose determinations and will be generally referred to as the “experiential bolus assistant,” or “EBA” for short. However, it is stressed that these example embodiments can be used with all types of insulin (e.g., long-acting insulin, intermediate, short-acting insulin, etc.), and other types of diabetes medications other than insulin. The example embodiments can also be used to determine types of dosages other than bolus dosages, such as basal dosages or basal time-varying dosage profiles, etc.

Before describing the embodiments in detail, it will be understood by those of skill in the art that any one or more of the steps of the example methods described herein can be stored as software instructions in a non-transitory memory of a reader device, a remote computing device, a trusted computer system, such as those described with respect to FIG. 1, or a drug delivery device. The stored instructions, when executed, can cause the processing circuitry of the associated device or computing system to perform any one or more of the steps of the example methods described herein. In some embodiments, the stored instructions can be implemented as one or more downloadable software applications (“an App”) on a reader device, such as a mobile telephone or smartphone, from which the software can communicate with a remote server (e.g., a cloud-based server), which can provide more comprehensive and robust analytics accessible by the individual on the same or a second computing device. In other embodiments, the stored instructions can be implemented as a web interface, accessible through a standard web browser, on a reader device or a computing system.

When used with an analyte monitoring system 100, these embodiments can capture, categorize, and index glucose responses to the meals and meal-time insulin doses (administered to compensate for the meal), and thus provide the user with additional data from which the user's insulin dosages can be perfected or “fine-tuned.” In addition, over time, the example embodiments can provide recommendations as to the titration of the bolus amount for each meal.

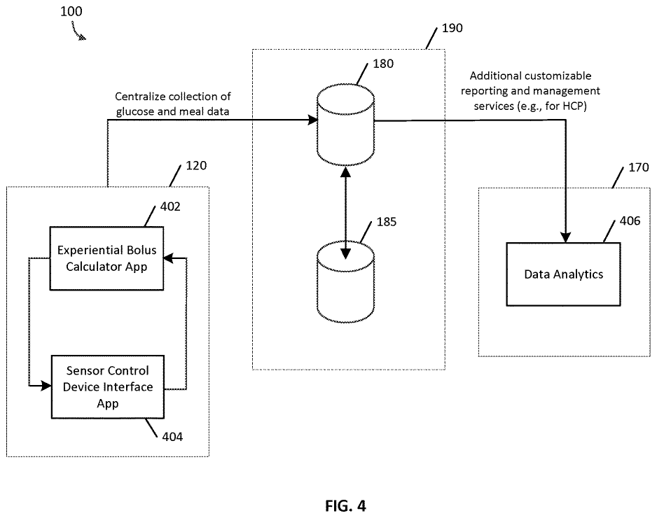

FIG. 4 is a block diagram depicting an example embodiment of system 100 configured to operate with the EBA in modular form. Here, EBA 402 is in the form of a downloadable app that has been downloaded (e.g., through an “app store” or equivalent), and installed on a smartphone 120. According to some embodiments, a second app 404 can also be downloaded and installed on smartphone 120, where the second app 404 is responsible for interfacing with sensor control device 102 (not shown), processing analyte data received therefrom, and configuring that data for display to the user. According to one aspect of some embodiments, app 404 can enable a commercial smart phone to serve as a reader device 120. While apps 402 and 404 are depicted in FIG. 4 as separate apps, they can also be combined into a single downloadable app (or module) with a single access icon on reader device 120.

According to another aspect of the embodiment, EBA 402 sends a request through a resident application programming interface (API) to app 404 for glucose data recently collected from the user. App 404 processes the request and supplies the queried data back to EBA 402, as shown in the loop depicted in FIG. 4. EBA 402 can associate in time the glucose data with the description of a recently consumed meal and, optionally, upload the meal and glucose data to trusted computer system 180 through network 190, represented here as a central cloud-based database. Medication dosages and/or post-prandial glucose data can also be uploaded to trusted computer system 180. The glucose, meal and medication dosage data can be categorized, indexed, and stored long-term as historical records in a database of central cloud system 180, and/or downloaded and stored long-term on reader device 120 or computing system 170. Nutrient parameters associated with a meal can also be stored for each historical record in central cloud-based database 180.

A user can access this data, for example, using a web browser operating on a smartphone 120, or via a separate computing device such as personal computer system 170, as shown in FIG. 4. According to some embodiments, the central cloud system 180 can also provide a data analytics tool via the user's web interface 406, which the user can use to enter user-specific information, adjust settings of the EBA, analyze glucose responses to meals consumed, and make informed decisions as to insulin dose adjustments and/or corrections. Furthermore, this data can be accessed directly by the user's HCP, either alone or in a collaborative fashion with the user during a visit, to investigate the efficacy of the user's insulin treatment and to make adjustments thereto. In some embodiments, computing device 170 can also be used to input meal information by the user. Overall, the analytics tool 406 can assist the user in long-term diabetes management and integration with other therapy decisions or user engagement systems.

Referring still to FIG. 4, central cloud system 180 can access a nutrition database system 185, which, according to one aspect of the embodiments, includes nutritional parameters associated with various meals and meal components. Central cloud system 180 and nutrition database system 185 can communicate over network 190, which can be over a local area network, a wide area network, over the internet, or over any similar communications network. According to one aspect of the embodiments, central cloud system 180 and nutrition database system 185 can be hosted at the same geographical location (i.e., where both systems can be managed by the same entity), or at different geographical locations (i.e., where nutrition database system 185 is managed by a third party). Those of skill in the art will also appreciate that central cloud system 180 and nutrition database system 185 can also be implemented as separate physical servers or separate instances of virtual machines on the same physical server. In addition, although FIG. 4 depicts nutrition database system 185 within network 190, according to some embodiments, nutrition database system 185 can reside on trusted computer system 170, and, optionally, apps 402 and 404 can communicate directly with trusted computer system 170 for communicating, transmitting and receiving updates, data, and reports.

According to one aspect of the embodiments, nutrition database system 185 can include an interface through which meal information is received as input, and from which nutritional parameters associated with the inputted meal information are outputted. In some embodiments, the nutritional parameters can include a carbohydrate parameter, a fat parameter, and/or a protein parameter, where each of the nutritional parameters are associated with the nutritional content of the inputted meal. Those of skill in the art will recognize that other nutritional parameters can be utilized, and are fully within the scope of the present disclosure.

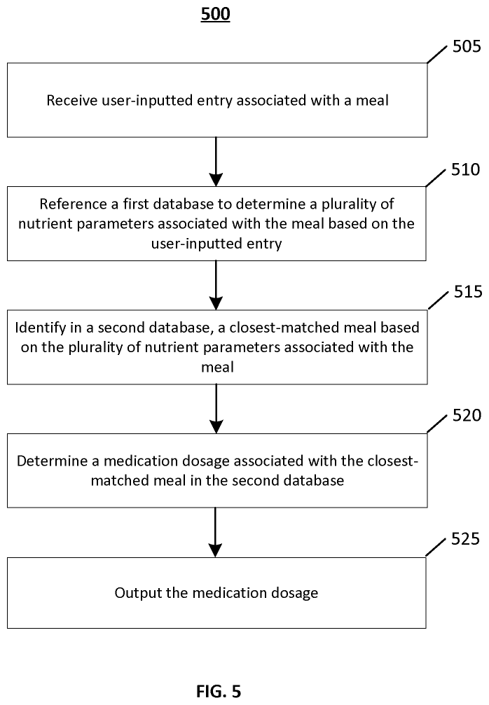

FIG. 5 is a flow chart depicting an example embodiment of a method for determining a medication dosage to be administered with the consumption of a meal, in which the method can be implemented, for example, via system 100 of FIG. 4. Beginning with Step 505, a user-inputted entry associated with a meal is received by system 100. According to some embodiments, the meal entry can be inputted through an app 402 or web-interface (e.g., via a web browser) on a reader device 120, such as a smartphone. In other embodiments, the meal entry can be inputted through a computing device 170, such as a personal desktop or laptop computer. According to some embodiments, the user-inputted entry can be a text entry, for example, provided in a “natural language” format, that can be descriptive of the meal being consumed. In still other embodiments, the user-inputted entry can be in the form of a photograph of the meal being consumed. Those of skill in the art will appreciate that other similar methods of user input (e.g., dropdown menus, selectable fields, check boxes, radio buttons, voice input, etc.) can be utilized and are within the scope of the present disclosure.

At Step 510, a first database is referenced to determine a plurality of nutrient parameters associated with the meal based on the user-inputted meal entry. According to one aspect of the embodiments, the first database can be a nutrition database system 185 (as shown in FIG. 4), and the plurality of nutrient parameters can include, for example, a carbohydrate parameter, a fat parameter, and/or a protein parameter for the meal associated with the user-inputted entry. Additionally, in some embodiments, prior to referencing the first database, the user-inputted entry can be transmitted from the reader device 120 or computing device 170 to the central cloud system 180. Central cloud system 180 can then reference the first database 185, using the user-inputted entry, to receive the associated nutrient parameters. In other embodiments, the user-inputted entry can be transmitted directly to first database 185 by the reader device 120 or computing device 170 and, subsequently, the first database 185 can transmit the associated nutrient parameters to the central cloud system 180. Optionally, at least a portion of the first database, which can include the nutrient parameters associated with the user-inputted meal entry, can be downloaded to and stored in association with the user-inputted meal entry on any of the central cloud system 180, reader device 120 and/or computing device 170. In still other embodiments, first database 185 can reside on trusted computing device 170, and the user-inputted entry can be transmitted from the reader device 120 to the trusted computing device 170, or inputted directly to the same trusted computing device 170 on which the first database 185 resides, without communicating with central cloud system 180 at this step.

Referring still to FIG. 5, at Step 515, a closest-matched meal is identified in a second database using the nutrient parameters associated with the meal. In many embodiments, the second database can be hosted on the central cloud system 180. In other embodiments, the second database can be located on reader device 120 and/or computing device 170.

According to one aspect of the embodiments, the closest-matched meal can be a historical meal record in the second database having a set of associated nutrient parameters that most closely resembles the nutrient parameters associated with the user-inputted meal. This can be determined, for example, by calculating a weighted set of differences between the nutrient parameters for each historical meal record and the nutrient parameters of the user-inputted meal entry, and selecting the historical meal record with the lowest total difference. To illustrate, in one example embodiment, the best-matched meal can be determined by calculating the lowest total difference resulting from the following equation: 0.5 *(absolute % difference in carbohydrates)+0.25 *(absolute % difference in fat)+0.25 *(absolute % difference in protein), where the “absolute % difference” can be the absolute value of the percentage difference between the nutrient parameter of the historical meal record and the nutrient parameter of the user-inputted meal entry. Those of skill in the art will recognize that other weighting factors can be used for each of the nutrient parameters. Likewise, the lowest total difference can also be calculated without using any weighting factors. In some embodiments, after the closest-matched meal is identified in the second database, a new historical meal record can be created in the second database for the user-inputted meal entry and subsequently linked to the closest-matched meal.

At Step 520, a medication dosage associated with the closest-matched meal in the second database is determined. In some embodiments, the medication dosage can be, for example, the most recent insulin dosage administered with the consumption of the closest-matched meal (that was recorded in the second database). In other embodiments, the medication dosage can be an average of the prior insulin dosages administered for all or a predetermined number of past instances where the closest-matched meal was consumed. In still other embodiments, the medication dosage can be an insulin dosage that is flagged in the second database as an optimal medication dosage for the closest-matched meal. Optionally, the determined medication dosage and/or the associated nutrient parameters can be stored in the second database with a historical record associated with the user-inputted meal entry. Finally, at Step 525, the determined medication dosage can be visually outputted to, for example, a display of the reader device 120 and/or a display of computing device 170.

### Example Embodiments for Identifying a Set of Meal Start Candidates and Meal Peak Response Candidates

### Example Characterizations of User-Initiated Analyte Checks

Some of the embodiments disclosed herein utilize analyte data from an analyte monitoring system, such as that described with respect to FIG. 1, in combination with information relating to user-initiated analyte checks, to determine a set of meal start candidates and meal peak response candidates. As described earlier, one of the challenges, with respect to analyte monitoring systems, is to be able to accurately correlate an individual's analyte data with the individual's meal consumption, as well as the individual's pre-prandial and post-prandial responses. This correlation can be useful in many applications, such as, for example, guidance for medication dosage titration. Unlike prior systems, the embodiments described herein do not rely solely upon manual blood glucose measurements or an individual's manual logging of meals, both of which can be both unrealistic and difficult for an individual to sustain. Before describing the embodiments in detail, however, it is desirable to describe certain aspects relating to user-initiated analyte checks and meal start times, all of which are relevant to the embodiments described herein.

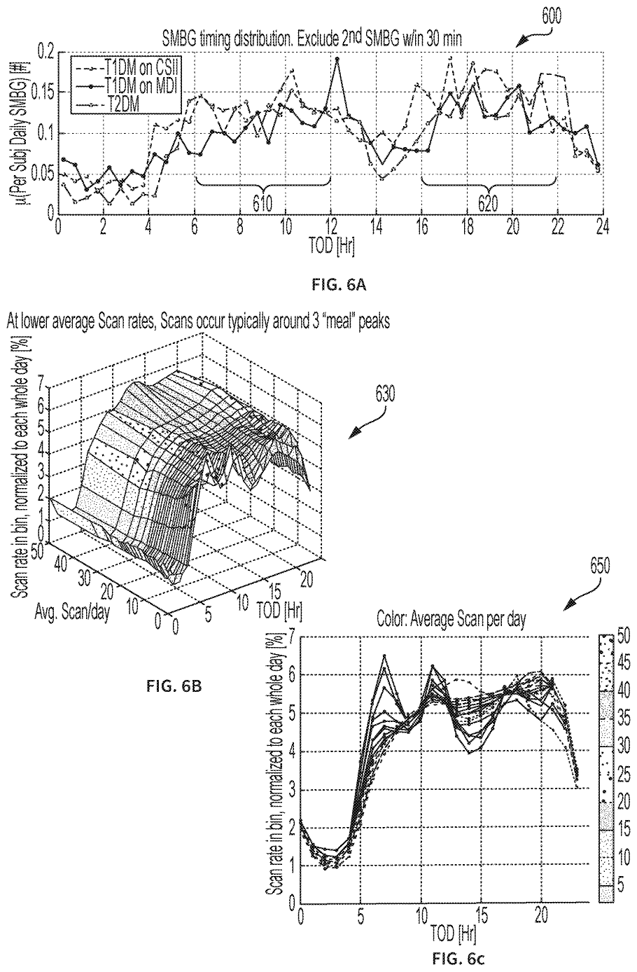

FIG. 6A is a graph 600 depicting a time-of-day (TOD) distribution of one type of user-initiated analyte check. In particular, graph 600 depicts self-monitoring blood glucose (SMBG) measurements from various sub-populations of a sensor study on insulin using patients. Here, the SMBG measurements consist of finger stick blood glucose tests. According to one aspect of graph 600, a distribution of SMBG measurements can be characterized by two data clusters 610, 620. A first cluster 610 is comprised of two weak modes which include pre-breakfast and pre-lunch SMBG measurements. In addition, a second cluster 620 is comprised of two slightly more distinct modes which include pre-dinner and pre-bedtime measurements. From this data, a reasonable inference can be drawn that meal start times correspond to most of the SMBG instances.

FIGS. 6B and 6C are graphs 630 and 650, respectively, both of which depict distributions of another type of user-initiated analyte checks. In particular, graphs 630 and 650 depict distributions of sensor scan instances from an analyte reader device for a large, de-identified population database of analyte reader device users. According to one aspect of graphs 630 and 650, the distributions of sensor scan instances are plotted as a function of time-of-day and average scans per day. Similar to the SMBG distribution of FIG. 6A, the sensor scan instance distributions in FIGS. 6B and 6C show that, at the lower average scans-per-day range, the sensor scan instance distribution is characterized by peaks similar to those of the SMBG measurement distribution of FIG. 6A. That is, meal start times also correspond to most of the sensor scan instances.

Although FIGS. 6A to 6C depict distributions for specific types of user-initiated analyte checks, those of skill in the art can reasonably infer that similar distributions occur with other types of user-initiated analyte checks, such as sensor viewer usage instances on a smartphone or receiver display activation instances in a continuous glucose monitoring (CGM) system.

### Examples for Determining Time Derivatives and Acceleration Characteristics

In accordance with the embodiments described herein, it is also desirable to describe certain characteristics of the analyte data of an analyte monitoring system, which can be utilized by the embodiments to identify a set of meal start candidates and meal peak response candidates.

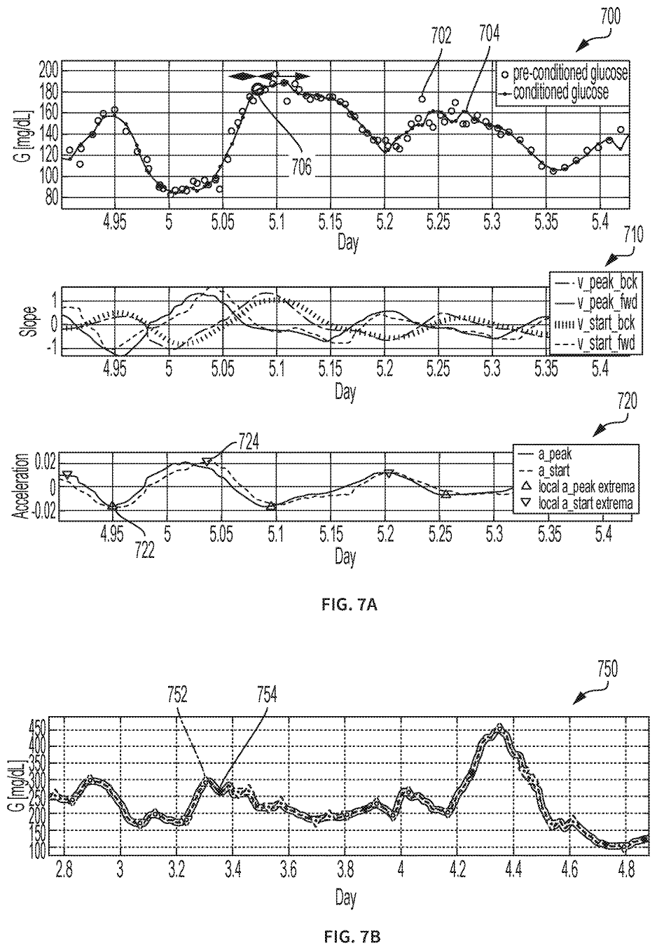

FIG. 7A depicts three graphs 700, 710, and 720, each of which illustrate certain characteristics relating to a sample set of analyte data, e.g., blood glucose concentration data, from an analyte monitoring system. Referring first to upper graph 700, as indicated by the y-axis, data points 702 (white circles) correspond to an analyte concentration, e.g., a blood glucose concentration, over a time period of days, as indicated by the x-axis. Data points 702 can be raw data received from an analyte sensor, which may include irregularly spaced data points and/or questionable readings. In certain embodiments, after being received from the analyte sensor, data points 702 can be conditioned to remove questionable readings and to smooth the data, resulting in conditioned data points 704 (dark circles). Conditioned data points 704 can be characterized by regularly spaced glucose values. According to some embodiments, data conditioning can include determining whether sampled glucose data may be outliers when compared to other sampled glucose data that are close in temporal proximity. Further details regarding performing data conditioning and recovery are described in U.S. patent application Ser. No. 14/210,312, entitled “Noise Rejection Methods and Apparatus for Sparsely Sampled Analyte Sensor Data,” filed on Mar. 13, 2014, the disclosure of which is incorporated herein by reference for all purposes.

Referring still to FIG. 7A, middle and lower graphs 710 and 720, respectively, depict additional characteristics of the analyte data of graph 700. More specifically, middle graph 710 depicts multiple line plots of time derivatives, or slopes, of the analyte data from graph 700. According to one aspect of the embodiments, for each time instance, k, a pair of time derivatives associated with a meal peak response candidate can be calculated, and, likewise, a pair of time derivatives associated with a meal start candidate can be calculated. In particular, a pair of time derivatives can be calculated by computing a rate of change of analyte data in a forward time window and a rate of change of analyte data in a backward time window. For example, as can be seen in upper graph 700, if circled data point 706 occurs at time instance, k, then a forward time window is indicated by the double-sided arrow to the right of data point 706, and the backward time window is indicated by the double-sided arrow to the left of data point 706. A forward time window can be from the present measurement at instance, k, to its near future time instance, e.g., 2 to 3 hours later. Similarly, a backward time window includes using sampled glucose data in a backward time window, i.e., from the present measurement at instance, k, to its near past time instance, e.g., 1 to 2 hours prior. According to some embodiments, a time derivative, or slope, can then be determined by fitting a straight line through the analyte measurements within each respective time window using the Least-Squares error fit method.

According to one aspect of the embodiments, the forward time window associated with a meal start candidate does not necessarily have the same width as the forward time window associated with a meal peak response candidate. Similarly, the backward time window associated with a meal start candidate does not necessarily have the same width as the backward time window associated with a meal peak response candidate.

Referring back to graph 710 of FIG. 7A, multiple line plots comprising time derivatives are shown, each of which can be calculated according to the methods described above. In particular, a plot of the time derivatives for the forward rate of change associated with a meal peak response candidate is shown as v_peak_fwd(k). A plot of the time derivatives for the backward rate of change associated with a meal peak response candidate is shown as v_peak_bck(k). Similarly, a plot of the time derivatives for the forward rate of change associated with a meal start candidate is shown as v_start_fwd(k). A plot of the time derivatives for the backward rate of change associated with a meal start candidate is shown as v_start_bck(k).

Referring still to FIG. 7A, lower graph 720 depicts multiple line plots for acceleration derived from the time derivatives shown in middle graph 710. In particular, lower graph 720 shows acceleration associated with a meal peak response candidate, a_peak(k), where a_peak(k) is calculated as (v_peak_fwd(k)−v_peak_bck(k))/T_peak, and where T_peak is a pre-determined sample period scaling factor for an associated meal peak response candidate (e.g., 1 to 3 hours). Similarly, lower graph 720 depicts the acceleration associated with a meal start candidate, a_start(k), where a_start(k) is calculated as (v_start_fwd(k)−v_start_bck(k))/T_start, and where T_start is a pre-determined sample period scaling factor for an associated meal start candidate (e.g., 1 to 3 hours).

Referring still to lower graph 720 of FIG. 7A, an initial set of meal start candidates and meal peak response candidates can be identified by determining local optima of acceleration from the acceleration line plots. According to the embodiments, the local optima of acceleration can be identified based upon signal analysis to identify extreme bend points. For instance, at each time instance, k, any a_peak values that fall within either the forward time window or the backward time window, with the exception of the value at time instance, k, a_peak(k), are identified. If the value of a_peak(k) is less than or equal to the minimum a_peak values in the two aforementioned time windows, the current time instance, k, is determined as a meal peak response candidate. Similarly, at each time instance k, any a_start values that fall within either the forward time window or the backward time window, with the exception of the value at time instance, k, a_start(k), are identified. If the value of a_start(k) is greater than or equal to the maximum a_start values in the two aforementioned time windows, then the current time instance k is determined as a meal start candidate.

According to another aspect of the embodiments, if a time instance, k, has been previously identified as a meal peak response candidate, and is also identified as a meal start candidate, the meal start candidate tag is moved to the next instance k+1. Graph 720 illustrates the identification of local acceleration optima, i.e., the meal start candidates and meal peak response candidates, as indicated by “up” triangles 722 and “down” triangles 724, respectively.

Further details regarding the above calculations, including the determination of time derivatives and local optima of acceleration are described in U.S. Publication No. 2017/185748A1 (“the '748 Publication”), the disclosure of which is incorporated herein by reference for all purposes.

### Example Embodiments Utilizing Analyte Data and User-Initiated Analyte Checks to Identify Meal Start Candidates and Meal Peak Response Candidates

Example embodiments of systems, devices, and methods for determining a set of meal start candidates and meal peak response candidates based on user-initiated analyte checks and analyte data from an analyte monitoring system will now be described.

It will be understood by those of skill in the art that any one or more of the steps of the example methods described herein can be stored as software instructions in a non-transitory memory of a sensor control device, a reader device, a remote computer, or a trusted computer system, such as those described with respect to FIG. 1. The stored instructions, when executed, can cause the processing circuitry of the associated device or computing system to perform any one or more of the steps of the example methods described herein. It will also be understood by those of skill in the art that, in many of the embodiments, any one or more of the method steps described herein, including the calculation of time derivatives, acceleration, or local optima thereof, can be performed using real-time or near real-time sensor data. In other embodiments, any one or more of the method steps can be performed retrospectively with respect to stored sensor data. In some embodiments, the method steps described herein can be performed periodically, according to a predetermined schedule, and/or in batches of retrospective processes.

It will also be appreciated by those of skill in the art that the instructions can be stored in non-transitory memory on a single device (e.g., a sensor control device or a reader device) or, in the alternative, can be distributed across multiple discrete devices, which can be located in geographically dispersed locations (e.g., a cloud platform). Likewise, those of skill in the art will recognize that the representations of computing devices in the embodiments disclosed herein, such as those shown in FIG. 1, are intended to cover both physical devices and virtual devices (or “virtual machines”).

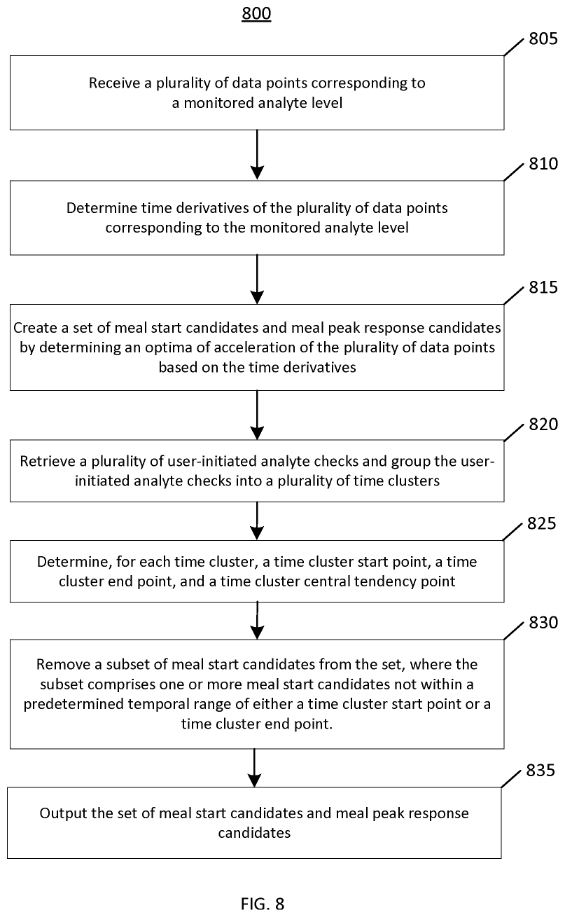

FIG. 8 is a flow diagram depicting an example embodiment of a method 800 for identifying a set of meal peak response candidates and meal start candidates. Beginning with Step 805, a plurality of data points corresponding to a monitored analyte level is received. According to some embodiments, the monitored analyte level can be a monitored blood glucose concentration. Those of skill in the art, however, will recognize that other analytes, such as acetyl choline, amylase, bilirubin, cholesterol, chorionic gonadotropin, HbAlc, creatine kinase (e.g., CK-MB), creatine, creatinine, DNA, fructosamine, glucose derivatives, glutamine, growth hormones, hormones, ketones, ketone bodies, lactate, peroxide, prostate-specific antigen, prothrombin, RNA, thyroid stimulating hormone, and troponin, as well as drugs, such as, for example, antibiotics (e.g., gentamicin, vancomycin, and the like), digitoxin, digoxin, drugs of abuse, theophylline, and warfarin, may also be monitored, and are fully within the scope of the present disclosure. According to some embodiments, the plurality of data points may be conditioned either before or after being received, as previously described above, to remove questionable readings, to smooth the plurality of data points, and/or to create regularly spaced analyte values.

Next, at Step 810, time derivatives for the plurality of data points corresponding to the monitored analyte level are determined. The time derivatives for the plurality of data points can be determined according to the calculations previously described with respect to graphs 700 and 710 of FIG. 7A. Subsequently, at Step 815, an initial set of meal start candidates and meal peak response candidates is created by determining local optima of acceleration of the plurality of data points based on the time derivatives determined at Step 810. The local optima of acceleration can be determined according to the calculations previously described with respect to graph 720 of FIG. 7A.

At Step 820, a plurality of user-initiated analyte checks is retrieved and grouped into a plurality of time clusters. The user-initiated analyte checks can comprise one or more of finger stick blood glucose tests, sensor scan instances from an analyte reader device, sensor viewer usage instances on a smartphone, or receiver display activation instances in a continuous glucose monitoring (CGM) system. According to some embodiments, the plurality of time clusters can comprise a subset of user-initiated analyte checks within a predetermined period of minutes.

At Step 825, a time cluster start point, a time cluster end point, and a time cluster central tendency point for each time cluster is determined. In some embodiments, the time cluster central tendency point can be a mean, a median, or a mode.

At Step 830, a subset of meal start candidates is removed from the initial set of meal start candidates and meal peak response candidates. According to one aspect of the embodiments, the subset of meal start candidates can include one or more meal start candidates that are not within a predetermined temporal range of either a time cluster start point or a time cluster end point.

At Step 835, the modified set of meal start candidates and meal peak response candidates is outputted to the individual user. In some embodiments, the output can be in the form of a graphical user interface on the display of a reader device, such as a smart phone. In other embodiments, the output can be an auditory or vibratory signal that is outputted to a sensor control device, a reader device, a local computer, or a trusted computer system.

It will be understood by those of skill in the art that, although method 800 shows the retrieval, grouping, and time cluster analysis of user-initiated analyte checks at Steps 820 and 825, these steps can be performed prior to or concurrently with any of the other steps of method 800.

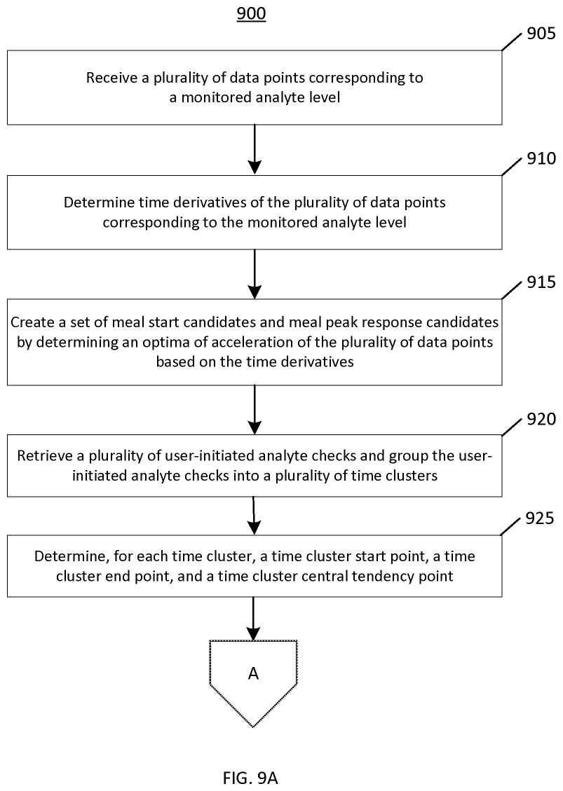

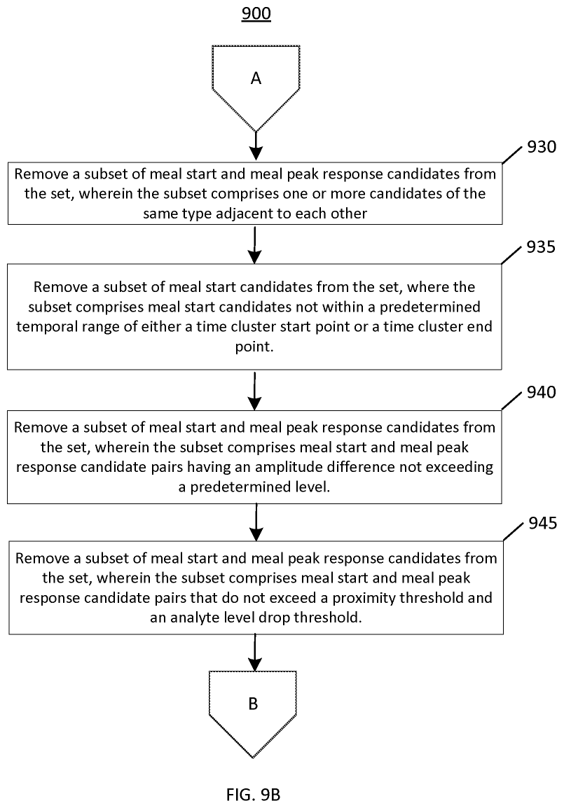

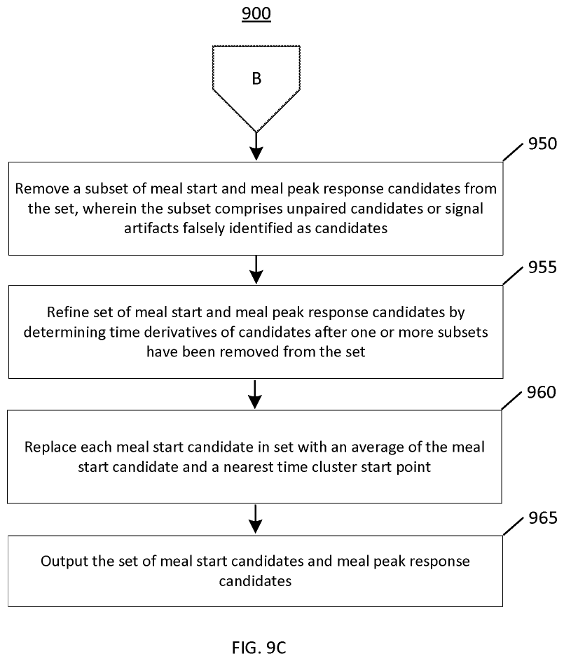

FIGS. 9A, 9B and 9C are flow diagrams depicting another example embodiment of a method 900 for identifying a set of meal peak response candidates and meal start candidates. Like previous method 800, method 900 is also based on user-initiated analyte checks and analyte data from an analyte monitoring system, but further includes additional steps of removing multiple subsets of meal start candidates and meal peak response candidates from the set, and further refining the set. Further details regarding these additional steps of removing multiple subsets of meal start candidates and meal peak response candidates are described in the '748 Publication, the disclosure of which is incorporated herein by reference for all purposes.

Referring first to FIG. 9A, Steps 905 to 925 of method 900 are the same as Steps 805 to 825 of method 800, and include receiving a plurality of data points, determining time derivatives, creating an initial set of meal start candidates and meal peak response candidates by determining local optima of acceleration, retrieving and grouping into time clusters a plurality of user-initiated analyte checks, and determining a time cluster start point, end point and central tendency point for each time cluster.

Turning to FIG. 9B, at Step 930, a subset of meal start candidates and meal peak response candidates is removed from the initial set, where the subset comprises one or more meal start candidates and/or meal peak response candidates adjacent to candidates of the same type. Since a meal start event cannot be adjacent in time to another start event, and similarly, a meal peak response event cannot be adjacent in time to another peak response event, adjacent candidates of the same type are identified and removed from the set under consideration.

According to some embodiments, for example, a meal peak response candidate is removed from the initial set based on the following criteria: (1) the next instance in the set is also a meal peak response candidate; (2) the next instance in the set has a larger analyte value than the current instance; and (3) the rate from the forward peak calculation of the current instance is more than a non-negative noise floor v_min_rise (e.g. 0.5 mg/dL/min). Calculated rates of change whose absolute numbers are close to zero tend to contain a lot of noise. Additionally, in certain embodiments, a meal peak response candidate is also removed if the previous instance in the set is also a meal peak response candidate, and the previous instance in the set has a larger analyte value than the current instance.

Similarly, in certain embodiments, meal start candidates are removed because the previous instance of an adjacent meal start candidate has a smaller analyte value. That is, a meal start candidate is removed based on the following criteria: (1) the previous instance in the set is also a meal start candidate; (2) the previous instance in the set has a smaller analyte value than the current instance; and (3) the value a_start(m−1) is smaller than a_start(m), where m is the current instance. In addition, in certain embodiments, a meal start candidate is also removed if the next instance in the set is also a meal start candidate, and the next instance has an analyte value that is either equal to or less than the analyte value of the current instance.

Referring again to FIG. 9B, Step 935 of method 900 is the same as Step 830 of method 800, and includes removing a subset of meal start candidates from the set, where the subset comprises one or more meal start candidates not within a predetermined temporal range of either a time cluster start point or a time cluster end point.

At Step 940, another subset of meal start candidates and meal peak response candidates is removed from the set, where the subset includes meal start candidate and meal peak response candidate pairs, and where each pair has an amplitude difference that does not exceed a predetermined level. More specifically, in certain embodiments, meal peak response candidates in the set are analyzed to determine whether the change in analyte value from the previous instance, which would be a meal start candidate, to the current meal peak response candidate is sufficiently large. In other words, a current meal peak response candidate is removed from the set when the following criteria are met: (1) previous instance, m−1, in the set is a meal start candidate; (2) the current instance, m, is a meal peak response candidate; and (3) the difference between the amplitude of m and the amplitude of m−1 is less than or equal to a predetermined minimum amplitude. Moreover, in certain embodiments, when a meal peak response candidate is removed under these conditions, the corresponding meal start candidate, m−1, is also removed.

At Step 945, another subset of meal start candidates and meal peak response candidates is removed from the set, where the subset includes meal start candidate and meal peak response candidate pairs, and where each pair does not exceed a proximity threshold and an analyte level drop threshold. That is, in certain embodiments, meal start candidates that are too close in time to a prior meal peak response candidate, and whose value is not significantly lower than the value of its prior meal peak response candidate, are removed from the set. More specifically, in certain embodiments, a meal start candidate at instance, m, is removed when the following criteria are met: (1) the previous instance, m−1, is a meal peak response candidate; (2) the current instance, m, is a meal start candidate; (3) the next instance, m+1, is a meal peak response candidate; (4) the average value of v_start_bck(m) and v_peak_fwd(m−1) is greater than a maximum post-prandial recovery descent rate, v_max_descent (e.g., ¼ mg/dL/min); and (5) the difference between the value of the current instance, m, and the previous instance, m−1, is less than or equal to a minimum required drop from a previous peak, g_min_drop (e.g., 5-10 mg/dL). Moreover, when these criteria are met and a meal start candidate is removed, the meal peak response candidate at the previous instance, m−1, is also removed.

Turning to FIG. 9C, at Step 950, another subset of meal start candidates and meal peak response candidates is removed from the set, where the subset includes unpaired meal start candidates or meal peak response candidates, or signal artifacts falsely identified as either meal start candidates or meal peak response candidates. According to one aspect of the embodiments, surviving spike artifacts might happen if, for example, data conditioning does not completely remove all artifacts. In certain embodiments, surviving spike artifacts falsely identified as meal start and meal peak response candidate pairs are removed from the set. More specifically, in certain embodiments, a current meal start candidate at instance, m, is removed from the set if: (1) the current instance, m, is a meal start candidate; (2) the next instance, m+1, is a meal peak response candidate; and (3) the aggregate rate of change, as calculated from g(m+1)−g(m), divided by the time interval between the two instances, m+1 and m, is larger than a maximum allowable initial post-prandial rate of change, v_max_initialSpike (e.g., 6 mg/dL/min, which is a rate of change that is likely not sustainable between two candidate points).

At Step 955, the resulting set of meal start candidates and meal peak response candidates can be further refined. Occasionally, because of the magnitude and asymmetrical nature of the forward and backward time windows used to calculate the time derivatives, and because some post-prandial responses may be followed by a subsequent post-prandial response without sufficient time for the original post-prandial response to revert to a baseline, the identification of meal start candidates and meal peak response candidates may be slightly biased before or after the likely instances. Further refinement of the set, after removal of the aforementioned subsets, can be performed to address these circumstances.

According to one aspect of the embodiments, for each sampled analyte data at instance, k, g(k), an available sample that is as close to 30 minutes prior to k as possible, g_prev(k), is identified. Also, for each sampled analyte data at instance, k, g(k), an available sample that is as close to 30 minutes after k as possible, g_after(k), is identified. Then, forward and backward slopes, v_fwd(k) and v_bck(k) are determined by taking the difference, g_after(k)−g(k), and dividing it by their time interval (e.g., 30 minutes). Likewise, backward slope, v_bck(k), is calculated by taking the difference, g(k)−g_prev(k), and dividing it by their time interval. The difference in slope, dv(k), is determined by taking the difference v_fwd(k)−v_bck(k). Those of skill in the art will recognize that analyte data samples of different time durations besides 30 minutes can be utilized (e.g., 15 minutes, 60 minutes, 90 minutes, etc.), and are fully within the scope of the present disclosure.

Subsequently, meal start and meal peak response candidate pairs from the set are analyzed according to the following steps. For each start and peak pair, an analyte time series, g_array_start, up to 90 minutes prior to the start candidate, and up to 60 minutes after the start candidate is defined. The defined analyte time series, g_array_start, includes the meal start candidate. Similarly, a glucose time series, g_array_peak, up to 60 minutes prior to the peak candidate, and up to 180 minutes after the peak candidate is defined. The analyte time series, g_array_peak, includes the peak candidate. Next, g_array_peak is “trimmed” of any sampled analyte data whose timestamp overlaps the start time of the next pair. For each value in g_array_start and g_array_peak, the corresponding differences in slope values, dv, are determined, and the arrays for these values, dv_array_start and dv_array_peak, are defined. Those of skill in the art will recognize that other time durations for g_array_start and g_array_peak (e.g., 30 minutes prior, 30 minutes after, 45 minutes prior, 45 minutes after, etc.) can be utilized and are fully within the scope of the present disclosure.

Thereafter, in certain embodiments, a subset of time instances is determined such that (1) the measured analyte values at these instances are greater than or equal to the 75th percentile of g_array_peak, and (2) the dv values at these instances are less than or equal to the 25th percentile of dv_array_peak. If such a subset contains data, then the highest analyte value in this subset, g_max, and its corresponding instance, is stored. Similarly, another subset of time instances is determined such that (1) the measured analyte value at these instances are less than or equal to the 25th percentile of g_array_start, and (2) the dv values at these instances are greater than or equal to the 75th percentile of dv_array_start. If such a subset contains data, then the lowest glucose value in this subset, g_min, and its corresponding instance, is stored. According to the embodiments, the corresponding peak and start candidates for this pair are replaced by g_max and g_min, respectively, if the following criteria are met: (1) g_min, and g_max exist and are finite; (2) g_min occurs prior to g_max; and (3) g_min is less than g_max. Those of skill in the art will also understand that the 75th and 25th percentiles utilized to determine, respectively, g_max and g_min are not meant to be limiting, and that other percentiles (e.g., 80th percentile, 20th percentile) are fully within the scope of the present disclosure.

Further details regarding the refinement of the set of meal start candidates and meal peak response candidates are described in the '748 Publication, the disclosure of which is incorporated herein by reference for all purposes

Referring again to FIG. 9C, after the set of meal start candidates and meal peak response candidates are refined, then at Step 960, each meal start candidate in the set can be replaced with an average of the meal start candidate and a nearest time cluster start point. At Step 965, the modified set of meal start candidates and meal peak response candidates is outputted to the user. In some embodiments, the output can be in the form of a graphical user interface on the display of a reader device, such as a smart phone. In other embodiments, the output can be an auditory or vibratory signal that is outputted to a sensor control device, a reader device, a local computer, or a trusted computer system.

Although FIGS. 9A to 9C depict discrete steps that are performed with respect to an initial set of meal start candidates and meal peak response candidates, those of skill in the art will appreciate that method 900 can exclude one or more steps. In some embodiments, for example, method 900 can exclude Step 965. In other words, after the refinement step is performed at Step 955, the set of meal start candidates and meal peak response candidates is outputted to the user at Step 965. In other embodiments, method 900 can exclude Step 935, where after a subset of adjacent candidates are removed at Step 930, the next step performed is the removal of a subset of candidates having an amplitude difference not exceeding a predetermined level. Those of skill in the art will also recognize that method 900 can include any of the described steps in any order or combination, and any such combinations or permutations of steps is fully within the scope of the present disclosure. Likewise, although method 900 shows the retrieval, grouping, and time cluster analysis of user-initiated analyte checks at Steps 920 and 925, those of skill in the art will appreciate that these steps can be performed prior to or concurrently with any of the other steps of method 900.

### Example Embodiments for Recommending a User-initiated Analyte Check

Some of the embodiments described herein can recommend a user-initiated analyte check in the future based on a current recorded action, if the recorded action corresponds to a historical user action associated with a glycemic risk. As previously described, analyte monitoring systems can provide for a more robust and convenient way of tracking an individual's physiological responses. For example, analyte monitoring systems can include a sensor control device that is worn on an individual's body, and which can continuously collect analyte measurements and transfer data in response to a scan by a reader device (such as by using a Near Field Communication (NFC) or Radio Frequency Identification (RFID) protocol). One challenge with analyte monitoring systems, however, is that the increased influx of data may lead to user disengagement and, eventually, less frequent use by the individual patient. The embodiments described herein can increase engagement by the individual by suggesting useful instances to perform user-initiated analyte checks (e.g., scans). In this manner, the embodiments may help to mitigate certain glycemic risks, such as, for example, hypoglycemia or hyperglycemia.

Before describing the embodiments, as with many of the previous embodiments, it will be understood by those of skill in the art that any one or more of the steps of the example methods described herein can be stored as software instructions in a non-transitory memory of a sensor control device, a reader device, a remote computer, or a trusted computer system, such as those described with respect to FIG. 1. The stored instructions, when executed, can cause the processing circuitry of the associated device or computing system to perform any one or more of the steps of the example methods described herein. It will also be understood by those of skill in the art that, in many of the embodiments, any one or more of the method steps described herein can be performed using real-time or near real-time sensor data. In other embodiments, any one or more of the method steps can be performed retrospectively with respect to stored sensor data. In some embodiments, the method steps described herein can be performed periodically, according to a predetermined schedule, and/or in batches of retrospective processes.

It will also be appreciated by those of skill in the art that the instructions can be stored in non-transitory memory on a single device (e.g., a reader device) or, in the alternative, can be distributed across multiple discrete devices, which can be located in geographically dispersed locations (e.g., a cloud platform). Likewise, those of skill in the art will recognize that the representations of computing devices in the embodiments disclosed herein, such as those shown in FIG. 1, are intended to cover both physical devices and virtual devices (or “virtual machines”).

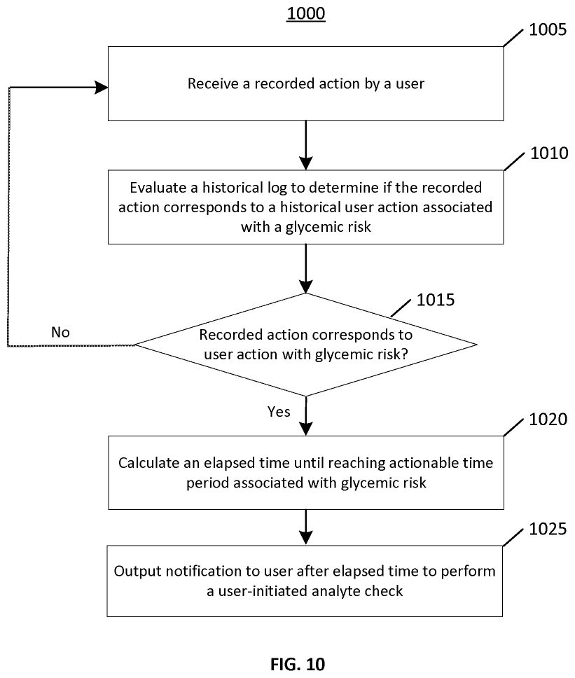

FIG. 10 is a flow diagram depicting an example embodiment of a method 1000 for recommending a user-initiated analyte check. According to one aspect of the embodiments, the user-initiated analyte check can be one or more of a finger stick blood glucose test, a sensor scan instance from a reader device, a sensor viewer usage instance on a smartphone, or a receiver display activation instance in a continuous glucose monitoring (CGM) system. Beginning with Step 1005, a recorded action by a user is received. According to some embodiments, the recorded action by the user can be the entry of a carbohydrate amount, the application of a medication, or the use of a bolus calculator, e.g., to correct glucose to a target glucose value. At Step 1010, a historical log is evaluated to determine if the current recorded action corresponds to a historical user action associated with a glycemic risk, such as, e.g., a hypoglycemic risk or a hyperglycemic risk. According to some embodiments, for example, evaluating the historical log can include comparing a time of day of the recorded action with a time of day of the historical user action associated with a glycemic risk. In other embodiments, evaluation of the historical log can include evaluating similar inputs from similar times of day from past records, and assessing the glycemic impact of the similar past inputs.

By way of illustration, the recorded action can be a user utilizing a bolus calculator on a reader device, for example, to correct his or her blood glucose level to a target glucose value or range, where an insulin sensitivity factor is stored in the memory of the reader device. If the current insulin bolus target correction applied by the patient is equivalent to a significantly higher or lower insulin sensitivity factor than what had been previously used in the same meal period of the day (e.g., lunch), a higher risk of hypoglycemia or hyperglycemia is determined.

As another illustration, in some embodiments, trend uncertainty estimates can be used to determine if a trend-based insulin correction recommendation has a significant chance of resulting in hypoglycemia or hyperglycemia. If a trend estimate uncertainty exceeds a predetermined threshold, or if a risk calculation based on the trend uncertainty exceeds a predetermined threshold, then a glycemic risk is determined and a reminder to perform a user-initiated analyte check can be generated at some appropriate time in the future. The risk calculation may generally be dependent on one or more glucose readings and may not be explicitly dependent on a trend estimate.

As yet another example, another recorded action can be a user entering a carbohydrate amount that is abnormally large. In these circumstances, it is possible that the patient is adjusting the carbohydrate amount to account for extra macronutrients (e.g., protein and/or fat), or to account for a larger-than-usual meal. Because the post-prandial glucose excursion may be different from usual, a higher glycemic risk may be determined. As another example, another recorded action can be a user entering bolus insulin information or meal information into a bolus calculator or meal/medication logging application at a time of day that is significantly different from past logs. For example, due to unforeseen circumstances, the patient had a late lunch, or an earlier but smaller lunch. In those circumstances, it may be possible that the timing of the meal or insulin would result in a determination of a higher glycemic risk.

It should be noted, and will be apparent to those of skill in the art, that the above examples of evaluating a historical log to determine if a recorded action corresponds to a historical user action associated with a glycemic risk are merely illustrative and are not intended in any way to limit the scope of the embodiments.

More specifically, in some embodiments, the evaluation of the historical log can include retrieving an insulin sensitivity factor stored in memory, determining if an analyte trend uncertainty estimate exceeds a predetermined analyte trend threshold, or determining if a degree of glycemic risk exceeds a predetermined risk threshold.

At Step 1015, if it is determined that the recorded action does not correspond to a user action associated with a glycemic risk, then method 1000 returns to Step 1005. However, if the recorded action corresponds to a user action associated with a glycemic risk then, at Step 1020, a likely elapsed time until reaching an actionable time period associated with the glycemic risk is calculated. According to one aspect of the embodiments, the elapsed time can be a single instance in the near future (e.g., 65 minutes from now), or a set of instances (e.g., 65, 90, and 100 minutes from now). In some embodiments, after the elapsed time is calculated, the user can be prompted to confirm outputting a notification after the elapsed time.

Referring still to FIG. 10, at Step 1025, a notification to perform a user-initiated analyte check is output to the user after the elapsed time. According to some embodiments, outputting the notification to the user to perform a user-initiated analyte check can include outputting the notification multiple times at a predetermined interval. In other embodiments, the notification can be outputted to the user in a single instance. Additionally, in some embodiments, the output can be in the form of a graphical user interface on the display of a reader device, such as a smart phone, to remind the user to scan the sensor control unit. In other embodiments, the output can be an auditory or vibratory signal that is outputted to a sensor control device, a reader device, a local computer, or a trusted computer system.

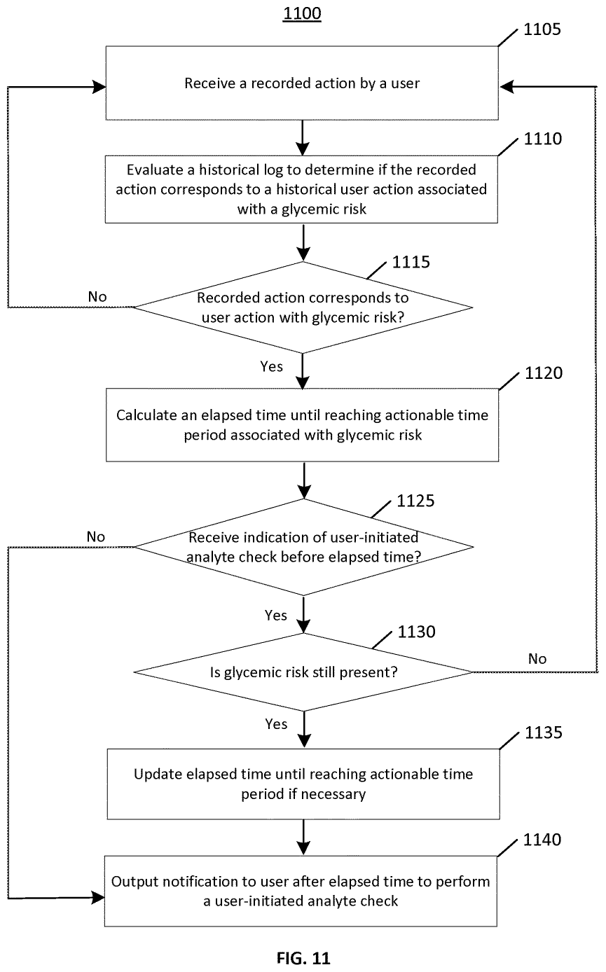

FIG. 11 is a flow diagram depicting another example embodiment of a method 1100 for recommending a user-initiated analyte check. In several regards, method 1100 is similar to method 1000. For instance, the first part of method 1100 (e.g., Steps 1105, 1110, 1115, and 1120) can be the same as the first part of previously described method 1000 (e.g., Steps 1005, 1010, 1015, and 1125). In addition, with respect to method 1100 of FIG. 11, after calculating the elapsed time until reaching an actionable time period associated with the glycemic risk, at Step 1125, method 1100 monitors for an indication of a user-initiated analyte check before the elapsed time. If no indication is received, then method 1100 proceeds to Step 1140, and a notification to perform a user-initiated analyte check is outputted to the user after the elapsed time. Similar to Step 1025 of method 1000 (FIG. 10), in some embodiments, the output can be in the form of a graphical user interface on the display of a reader device to remind the user to scan the sensor control unit. In other embodiments, the output can be an auditory or vibratory signal that is output to a sensor control device, a reader device, a local computer, or a trusted computer system.

If an indication of a user-initiated analyte check (e.g., sensor scan) is received before the elapsed time then, at Step 1130, data associated with the user-initiated analyte check is evaluated to determine whether the glycemic risk is still present. According to one aspect of the embodiments, the data associated with the user-initiated analyte check can be data indicative of a monitored analyte level, e.g., a blood glucose level.

If the glycemic risk is no longer present, then method 1100 returns to the beginning, or Step 1105. On the other hand, if the glycemic risk is determined to still be present at Step 1130, then, at Step 1135, the elapsed time until reaching the actionable time period is updated, if necessary. In some embodiments, for example, a second elapsed time until reaching a second actionable time period associated with the glycemic risk can be calculated. After the elapsed time (or second elapsed time) is reached then, at Step 1140, a notification to perform a user-initiated analyte check (or a second user-initiated analyte check) is outputted to the user. As with the previously described embodiments, outputting the notification to the user to perform a user-initiated analyte check can include outputting the notification multiple times at a predetermined interval, or in a single instance. In some embodiments, the output can be in the form of a graphical user interface on the display of a reader device, such as a smart phone, to remind the user to scan the sensor control unit. In other embodiments, the output can be an auditory or vibratory signal that is outputted to a sensor control device, a reader device, a local computer, or a trusted computer system.

Although the embodiments are described in terms of a monitored glucose level and glycemic risk, those of skill in the art will recognize that these embodiments can be utilized for other analytes, such as acetyl choline, amylase, bilirubin, cholesterol, chorionic gonadotropin, HbAlc, creatine kinase (e.g., CK-MB), creatine, creatinine, DNA, fructosamine, glucose derivatives, glutamine, growth hormones, hormones, ketones, ketone bodies, lactate, peroxide, prostate-specific antigen, prothrombin, RNA, thyroid stimulating hormone, and troponin, as well as drugs, such as, for example, antibiotics (e.g., gentamicin, vancomycin, and the like), digitoxin, digoxin, drugs of abuse, theophylline, and warfarin, may also be monitored, and are fully within the scope of the present disclosure.

For each and every embodiment of a method disclosed herein, systems and devices capable of performing each of those embodiments are covered within the scope of the present disclosure. For example, embodiments of sensor control devices are disclosed and these devices can have one or more analyte sensors, analyte monitoring circuits (e.g., an analog circuit), memories (e.g., for storing instructions), power sources, communication circuits, transmitters, receivers, clocks, counters, times, temperature sensors, processors (e.g., for executing instructions) that can perform any and all method steps or facilitate the execution of any and all method steps. These sensor control device embodiments can be used and can be capable of use to implement those steps performed by a sensor control device from any and all of the methods described herein. Similarly, embodiments of reader devices are disclosed, and these devices can have one or more memories (e.g., for storing instructions), power sources, communication circuits, transmitters, receivers, clocks, counters, times, and processors (e.g., for executing instructions) that can perform any and all method steps or facilitate the execution of any and all method steps. These reader device embodiments can be used and can be capable of use to implement those steps performed by a reader device from any and all of the methods described herein. Embodiments of computer devices and servers are disclosed, and these devices can have one or more memories (e.g., for storing instructions), power sources, communication circuits, transmitters, receivers, clocks, counters, times, and processors (e.g., for executing instructions) that can perform any and all method steps or facilitate the execution of any and all method steps. These reader device embodiments can be used and can be capable of use to implement those steps performed by a reader device from any and all of the methods described herein.

Computer program instructions for carrying out operations in accordance with the described subject matter may be written in any combination of one or more programming languages, including an object oriented programming language such as Java, JavaScript, Smalltalk, C++, C #, Transact-SQL, XML, PHP or the like and conventional procedural programming languages, such as the “C” programming language or similar programming languages. The program instructions may execute entirely on the user's computing device, partly on the user's computing device, as a stand-alone software package, partly on the user's computing device and partly on a remote computing device or entirely on the remote computing device or server. In the latter scenario, the remote computing device may be connected to the user's computing device through any type of network, including a local area network (LAN) or a wide area network (WAN), or the connection may be made to an external computer (for example, through the Internet using an Internet Service Provider).

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the foregoing description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

To the extent the embodiments disclosed herein include or operate in association with memory, storage, and/or computer readable media, then that memory, storage, and/or computer readable media are non-transitory. Accordingly, to the extent that memory, storage, and/or computer readable media are covered by one or more claims, then that memory, storage, and/or computer readable media is only non-transitory.

As used herein and in the appended claims, the singular forms “a,” “an,” and “the” include plural referents unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1-16. (canceled)

17. An electronic system configured to determine a medication dosage for administration with the consumption of a meal, the system comprising:
processing circuitry; and
a non-transitory memory comprising a plurality of instructions that, when executed, causes the processing circuitry to:
receive a user-inputted entry associated with the meal;
reference a first database to determine a plurality of nutrient parameters associated with the meal based on the user-inputted entry;
identify a closest-matched meal in a second database based on the plurality of nutrient parameters associated with the meal;
determine a medication dosage associated with the closest-matched meal in the second database; and
output the medication dosage.

18. The electronic system of claim 17, wherein the plurality of instructions, when executed, further causes the processing circuitry to store the user-inputted entry and the determined medication dosage in the second database.

19. The electronic system of claim 17, wherein the plurality of nutrient parameters includes a carbohydrate parameter, a fat parameter, and a protein parameter.

20. The electronic system of claim 17, wherein the second database includes a plurality of historical meal records, wherein each historical meal record comprises a plurality of stored nutrient parameters and a stored medication dosage.

21. The electronic system of claim 20, wherein the plurality of instructions, when executed, further causes the processing circuitry to determine a lowest total difference between the plurality of stored nutrient parameters for each historical meal record and the plurality of nutrient parameters associated with the meal.

22. The electronic system of claim 21, wherein the plurality of instructions, when executed, further causes the processing circuitry to calculate, for each historical record, an absolute percentage difference between each of the plurality of stored nutrient parameters and each of the plurality of nutrient parameters associated with the meal.

23. The electronic system of claim 22, wherein the plurality of instructions, when executed, further causes the processing circuitry to apply one or more weighted factors to the absolute percentage differences.

24. The electronic system of claim 23, wherein the plurality of nutrient parameters includes a carbohydrate parameter, a fat parameter, and a protein parameter, and wherein a weighted factor of the carbohydrate parameter is greater or equal to each of a weighted factor of the fat parameter and a weighted factor of the protein parameter.

25. The electronic system of claim 17, further comprising a web-interface configured to receive the user-inputted entry associated with the meal.

26. The electronic system of claim 17, further comprising a mobile app interface configured to receive the user-inputted entry associated with the meal.

27. The electronic system of claim 26, further comprising a smartphone.

28. The electronic system of claim 17, wherein the plurality of instructions, when executed, further causes the processing circuitry to download at least a portion of the first database to the non-transitory memory.

29. The electronic system of claim 17, wherein the second database is stored in the non-transitory memory.

30. The electronic system of claim 17, wherein the second database is stored on a location-independent server.

31. The electronic system of claim 17, wherein the plurality of instructions, when executed, further causes the processing circuitry to create a link between the closest-matched meal and the user-inputted entry in the second database.

32. The electronic system of claim 17, wherein the medication dosage comprises an insulin dosage.

33-96. (canceled)
